(19) United States
(12) Patent Application Publication
(10) Pub. No.: US 2009/0305358 A1
(43) Pub. Date: Dec. 10, 2009

(54) DNA Vector Production System

(76) Inventor: Yung-Nien Chang, Elkridge, MD (US)

Correspondence Address: Yung Nien Chang, 8048 Hillrise Court, Elkridge, MD, 21075, US

(21) Appl. No.: 11/921,842

(22) PCT Filed: Jul. 1, 2006
(86) PCT No.: PCT/US2006/025892

§ 371 Date: Dec. 10, 2007

Related U.S. Application Data
(60) Provisional application No. 60/696,052, filed on Jul. 1, 2005.
(60) Provisional application No. 60/722,348, filed on Oct. 1, 2005.

(51) Int. Cl. C12N 15/64; C12N 15/63; C12N 5/10; C12N 15/00

(52) U.S. Cl. 435/91.4; 435/320.1; 435/325; 435/455

(57) ABSTRACT

The invention discloses the production of double stranded DNA (dsDNA) vectors capable of delivering nucleic acids, including cDNA, antisense, ribozyme, and small interference RNA into cells. The invention also describes nucleic acid constructs as well as methods for the production of the dsDNA vectors.

### RELATED APPLICATIONS

This invention claims benefit of priority from U.S. Provisional Application 60/696,052, filed Jul. 1, 2005, and 60/722,348, filed Oct. 1, 2005, both of which are hereby incorporated by reference as if fully set forth.

### FIELD OF THE INVENTION

This invention relates to the production of double stranded DNA (dsDNA) vectors capable of delivering nucleic acids, including cDNA, antisense, ribozyme, small interference RNA, and micro RNA into cells. The invention provides nucleic acid constructs as well as methods for the production of such dsDNA vectors.

### BACKGROUND OF THE INVENTION

There are a number of means of delivering nucleic acids into cells. Nucleic acids can be introduced into cells by physical means such as by electroporation and by a gene gun that directly shoots gold particles coated with nucleic acids into cells. Nucleic acids can also be delivered into cells by chemical means such as by co-precipitation of nucleic acids with calcium phosphate or by liposome encapsulation. However, the most efficient method of delivering nucleic acids into organisms is by recombinant viral vectors. Viral vectors can infect and deliver foreign nucleic acids into cells as efficient as parental wild type viruses do. This is because a viral vector has been equipped with all the mechanisms of a wild type virus for efficient infection.

Recombinant viral vectors are genetically modified viruses. In some cases, and through genetic manipulations, these viruses are rendered replication deficient so that there will be no progeny virus produced after primary infection. With the exception of the viral nucleic acid sequences that are essential for viral genome replication and packaging, some or most of the viral genes that encode viral proteins that are essential for virus replication are removed from a viral genome in order to create a replication incompetent viral vector. These replication incompetent viral vectors are usually 1) produced from packaging cell lines that provide all the viral proteins that are essential to complement replication of viral vectors or 2) produced with helper viruses in case of AAV vector. Currently a number of viral vectors have been developed for gene transduction. They can be grouped as vectors that integrate into the host cell genome and non-integrating vectors.

Vectors that integrate are mainly made from retroviruses such as murine leukemia virus (MuLV) and lentiviruses (HIV-1, SIV, FIV, and BIV). These retroviral vectors can introduce and permanently integrate nucleic acids into host genomes. This type of vector may be used for long term expression of the delivered foreign nucleic acids in target cells. Where all the viral genes are deleted from retroviral vectors, these vectors may be almost non-immunogenic. The common disadvantage of retroviral vectors is their relatively low titer comparing to other vectors such as the adenoviral vectors. The titer of retroviral vector ranges from 10⁶ to 10⁷ transduction units (TU) per milliliter while adenoviral vector titer ranges from 10¹⁰ to 10¹³ TU/ml.

The non-integrating vectors include vectors developed from both RNA viruses such as alpha viruses (Sindbis virus, Semliki Forest virus, and et. al.) and DNA viruses such as adenovirus, adeno-associate virus (AAV), herpes simplex virus (HSV), and poxviruses (vaccinia virus). Both adenoviral vector and AAV vector are widely used non-integrating vectors. After entry into a cell, an adenoviral vector genome stays episomal, therefore expression of foreign nucleic acid in target cells may be transient. Although a wild type AAV specifically integrates into chromosome 19, an AAV vector was found not to integrate efficiently. Construction and plaque purification of recombinant adenoviral vector is a tedious process. Adenoviral vectors have been found to be very immunogenic. The host immune responses eliminate the transduced cells and also prohibit secondary transduction. AAV vectors were found less immunogenic. However, purification of AAV vector from the helper adenoviruses during vector production remains a great challenge.

Simian virus 40 (SV40) is a non-integrating DNA virus with a 5.2 kb double stranded circular DNA genome. It belongs to the Papovavirus family. SV40 based vectors have been developed for gene transfer and were found to efficiently transduce many eukaryotic tissues such as liver, lung, colon, spleen, brain, skin, kidney, and blood cells. One of the receptors for SV40 virus entrance is MHC-1 that is expressed on the cell surfaces of almost all mammalian cells. The SV40 vector was found not immunogenic in animal studies.

SV40 vectors can be produced by an in vitro packaging method and by a conventional method. A circular SV40 vector DNA that contains the SV40 replication origin and the encapsidation signal sequence can be packaged into virion particles in vitro by mixing vector DNA with baculovirus-expressed SV40 capsid proteins. Conventionally, an SV40 based vector is constructed by deleting the viral DNA sequence that encodes T antigen. Deletion of T antigen renders virus replication deficient. The SV40 viral DNA with the T antigen deleted was first cloned into a plasmid such as pBR322 to make an SV40 cloning vector. The cloning vector plasmid DNA can be amplified in bacteria. A transgene can be then cloned downstream of the SV40 early promoter of the cloning vector. To produce viral vector the viral DNA sequence has to be excised and purified from the cloning vector and be religated to form a circular DNA (see FIG. 1A). Viral vector can be produced and amplified by transfecting Cos-7 cells with the religated circular viral vector DNA (see FIG. 1A). Since Cos-7 cell line constitutively expresses T antigens that can complement the SV40 vector, Cos-7 cells can function as a packaging cell line for SV40 vector production. However, other cell lines can be used after the introduction of the gene for the SV40 large T-antigen.

The above is not intended as an admission that any is pertinent prior art. All statements are based on the information available and does not constitute any admission as to correctness.

### BRIEF SUMMARY OF THE INVENTION

The invention provides a more efficient means of producing vectors for delivery of nucleic acid molecules. The more efficient means may be viewed as a high through-put vector production system. In some embodiments, the system may be advantageously used in combination with small interfering RNA (siRNA) technology, such as in gene knockout or knock-down studies for drug screening. A high through-put double stranded DNA (dsDNA) vector production system provides a unique means to produce vectors for delivering small hairpin (shRNA), micro RNA, and other nucleic acids to a target cell for a variety of purposes, including drug screening as a non-limiting example.

In a first aspect, the invention provides nucleic acid constructs for the production of dsDNA vectors. In some embodiments, the dsDNA vector is SV40 based. Thus the invention includes an improved SV40-based vector production system as a non-limiting example. The nucleic acid constructs permit efficient introduction of other nucleic acid molecules into the dsDNA vector and in the production of high titer dsDNA vector particles.

The invention is based upon the use of and enzyme-mediated specific homologous recombination system. Thus any combination of target sequences and an enzymatic activity, such as a recombinase, that recombines them may be used in the practice of the invention. Non-limiting examples include the Cre-Lox system of site specific DNA recombination, which is described as an exemplification below; the FLP system, by use of the FLP recombinase (an enzyme native to the 2 micron plasmid of S. cerevisiae) active at a specific 34 base pair DNA sequence (termed the FLP recombinase target sequence); and the XerD recombinase based system, where XerD belongs to the lambda integrase family of enzymes.

The Cre-Lox system is described in U.S. Pat. No. 4,959,317 (the '317 patent), which is hereby incorporated by reference as if fully set forth. Briefly, the Cre-Lox system is based upon the use of the cre gene product, which is a site specific DNA recombinase. The CRE protein recombines DNA based upon the location of specific 34 basepair long sequences sites known as loxP sequences. A larger DNA molecule containing an internal sequence flanked by loxP sequences undergoes CRE protein mediated recombination to result in excision and religation of the flanked internal sequence into a circular DNA molecule containing one copy of the loxP sequence. The larger DNA molecule is also religated to retain one copy of the loxP sequence but loss of the excised internal sequence. The invention may be practiced with use of various cre and loxP sequences as provided by the '317 patent and as known in the art.

The invention thus provides a first nucleic acid molecule or construct that contains an internal dsDNA viral vector sequence flanked by recombination target sequences, such as loxP sequences. The internal dsDNA viral vector sequence is thus capable of being excised and circularized to form a dsDNA vector molecule with one copy of the target sequence. The first nucleic acid molecule or construct is optionally capable of expressing an enzymatic activity, such as the cre gene product, which facilitates the excision and circularization of the internal dsDNA viral vector sequence. The first nucleic acid molecule or construct thus includes a nucleic acid sequence in cis that is capable of expressing the recombinase activity for the production of the dsDNA vector.

In other embodiments, the first nucleic acid molecule or construct is not capable of expressing the enzymatic activity. Instead, the activity, like the cre gene product, is provided in trans by a second nucleic acid molecule or construct capable of expressing the recombinase activity. The second nucleic acid molecule may be present in the genome of a cell containing the first nucleic acid molecule. Alternatively, the second nucleic acid molecule or construct may be present as all or part of an episomal molecule in a cell containing the first nuclei acid molecule.

A dsDNA viral vector is derived from a dsDNA virus which is composed of nucleic acid sequences necessary for viral replication. The sequences are divided into those which are needed in cis or in trans. Sequences needed in cis are those which must be present on the virus, and thus viral vector, for replication. Sequences needed in trans are those which encode a gene product (or trans acting factor) which is needed for viral, and thus vector, replication. Sequences needed in trans may thus be present on a non-vector nucleic acid that is capable of being expressed to provide the gene product(s) needed for replication. The dsDNA viral vectors of the invention comprise cis acting viral sequences needed for vector replication. Non-limiting examples of such sequences include a viral origin of replication and a packaging or encapsidation signal. The former allows for the replication of the vector sequence while the latter allows the production of virion particles containing the vector.

Sequences needed in trans for vector replication may be provided by a second nucleic acid construct of the invention (optionally including sequences which provide the recombinase activity). The second nucleic acid construct may thus be viewed as a helper construct for replication of the viral vector. In some embodiments, the second construct is present in a cell used for vector production, such as by being integrated into the cell's genome or by being an episomally maintained molecule. Such a cell is a packaging cell of the invention, which optionally contains a first nucleic acid construct of the invention. A packaging cell comprises sequences to facilitate viral vector production and functions to complement, replicate, and package the viral vector constructs of the invention.

Alternatively, some or all of the trans acting sequences may be present on the vector containing constructs of the invention. They may thus be part of the vector sequence to be produced and/or packaged or part of the non-viral sequence in a first nucleic acid construct of the invention.

In some embodiments, the dsDNA viral vectors of the invention comprise a sequence which allows the vector to be capable of expressing a non-viral, or heterologous sequence. This sequence may be viewed as a “pay-load” to be expressed or delivered by a vector of the invention. Non-limiting examples of non-viral, or heterologous, sequences include cDNAs, shRNAs, miRNAs, ribozymes, and antisense sequences.

The invention further provides a method of producing a dsDNA viral vector. Such a method comprises contacting a first nucleic acid construct of the invention with a cognate recombinase activity to excise and circularize the dsDNA viral vector sequence from the construct based on the target sequences. In some embodiments, the contacting occurs intracellularly, such as in a packaging cell of the invention. The recombinase activity may be encoded by a second nucleic acid construct in such a cell.

In other embodiments, the cell is capable of packaging said dsDNA viral vector into virion particles. Such a cell provides all the trans acting factors needed to for the production of virion particles.

The invention also provides a dsDNA viral vector comprising a recombinase target sequence, such as loxP. Such a vector is the product of the above described method of producing a viral vector. Of course such a vector may comprise a non-viral (heterologous) sequence as described herein. The invention further provides a virion particle comprising such a vector as well as a method of delivering a non-viral or heterologous sequence to a cell by using such a vector. Such a method comprises contacting a cell with the vector as a nucleic acid molecule or as a virion particle to introduce the vector into the cell.

While the invention is exemplified by reference and description of SV40 based viral vectors, other dsDNA viral vectors may also be used. Non-limiting examples include viruses of the family Papovaviridae, which naturally fall into two genera, Papillomavirus and Polyomavirus. Properties shared by these viruses are small size, nonenveloped virion, icosahedral capsid, double-stranded circular DNA genome, and use of a cell's nucleus as the site of multiplication. In some embodiments, the viral vector is based on a viral genome that is a circular DNA molecule while in other embodiments, the vector is based on a linear viral genome.

SV40 belongs to genus Polyomavirus. In this genus there are members that infect different host species. Viruses that infect human include BK virus (BKV) and JC virus (JCV). Viruses that infect monkeys include simian virus 40 (SV40), simian agent 12 (SA12), and lymphotropic papovavirus (LPV). Bovine polyoma virus (BPoV) infects cattle, rabbit kidney vacuolating virus (RKV) infect rabbits, polyoma and K infect mice, hamster papovavirus (HaPV) infects hamsters, and Budgerigar fledgling disease virus (BFDV) infects budgerigar.

Papillomaviruses are widely distributed in mammals. Viruses have been characterized from humans, cattle, rabbits, horses, dogs, sheep, elk, deer, and mice. An avian papillomavirus has been isolated from a chaffinch. A species may have several distinct papillomaviruses; there 25 human papillomavirus (HPV-1˜25) and 6 of cattle (BPV1-1˜6). Human papillomavirus usually cause warts in hosts, however, HPV-16 can cause cervical carcinoma.

Given the similarities in genomic structure, all of the above described viruses may be used as, or to derive, a viral vector of the invention and so may be used as a gene delivery vehicle of the invention using the recombination based technology as described herein. As would be recognized by the skilled person, a viral vector of the invention would include the necessary cis components from the viral genome for the vector to replicate and be packaged into viral particles.

The details of additional embodiments are set forth in the accompanying drawings and the description below. Other features, objects, and advantages of the embodiments will be apparent from the drawings and detailed description, and from the claims.

### BRIEF DESCRIPTION OF THE FIGURES

FIG. 1 shows comparison between conventional means and the disclosed invention, exemplified by an SV40 vector production system as a non-limiting example. FIG. 1A shows the conventional SV40 vector production method. FIGS. 1B and 1C illustrates the disclosed invention using Cre-Lox mediated homologous recombination to produce SV40 viral vectors. FIG. 1B illustrates a system wherein the CRE recombinase activity is provided in trans (in a cell identified as Cos7-Cre) relative to the SV40 vector to be produced. FIG. 1C illustrates a system wherein the recombinase activity is encoded in cis. Cos-7 cells are indicated to show that the SV40 T antigen may be provided in trans.

FIGS. 2 to 4 show the flow chart of constructing SV40 vectors pSV-Ori, pSV, and pSV-Cre. pSV-Ori contains the SV40 origin of replication (Ori). pSV is similar to pSV-Ori except that additional sequences encoding, and capable of expressing, SV40 proteins are present. pSV-Cre is similar to pSV except that a sequence capable of expressing CRE recombinase activity is present in cis.

FIG. 5 shows cloning of a non-SV40 cDNA (exemplified by the eGFP cDNA as a non-limiting example) and/or a Pol III promoter (such as H1) into SV40 vectors (pSV or pSV-Cre).

FIG. 6 shows an SV40 vector DNA recombined into two circular DNAs by Cre-mediated site specific recombination. One circular DNA contains the SV40 sequence and can be packaged into virion particles. A transgene (eGFP) is juxtaposed immediate downstream of and expressed from the SV40 early promoter as a non-limiting example. The other circular DNA contains the plasmid backbone which previously contained the SV40 sequence. This circular DNA can be lost after additional cell divisions.

FIG. 7 shows SV40 vectors encoding shRNA capable of being expressed from the T7 promoter.

FIG. 8 shows the construction of an SV40 “gut-less” vector pSV2 (containing very few SV40 viral sequences). An SV40 DNA fragment that encompasses both the early and late gene polyadenylation signals (indicated by arrows) is cloned NotI and NheI sites of pSV-Ori to make the gut-less vector pSV2.

FIG. 9 shows the construction of an SV40 vector helper construct, pC-VP-SvNeo. The construct contains all the SV40 structural genes capable of being expressed under the control of the CMV promoter.

FIG. 10 shows cell lines Cos7-VP and Cos7-Cre-VP that are constructed by stably integrating pC-VP-SvNeo into Cos-7 and Cos7-Cre cells, respectively. These cells may be used in the production of vectors of the invention.

### DETAILED DESCRIPTION OF MODES OF PRACTICING THE INVENTION

In some embodiments of the invention, a nucleic acid construct (molecule) comprising a dsDNA viral vector sequence flanked by target sequences of an enzyme-mediated sequence-specific homologous recombination system, such as loxP, is disclosed. The viral vector is thus flanked by two sequences that are recognized by a recombinase activity which is capable of recombining, or ligating and rejoining, the two sequences to result in a circular molecule containing the viral vector and one copy of the target sequence. Non-limiting examples of the target sequences include loxP, sequences recombined by the FLP recombinase, and sequences recombined by the XerD recombinase. Of course any combination of target sequences and the cognate recombinase activity that recombines them, as known to the skilled person, may be used in the practice of the invention.

The nucleic acid construct is optionally isolated from other nucleic acid molecules or proteins used in its production. In some embodiments, the construct is in a solvent and at least about 50% by weight of the non-solvent molecules present with the construct. In additional embodiments, the construct is at least about 60%, at least about 70%, at least about 80%, at least about 90%, at least about 95%, or at least about 99% relative to non-solvent molecules. In further embodiments, the construct is at least about 50%, about 60%, about 70%, about 80%, about 90%, about 95%, or about 99% by weight of a solid composition.

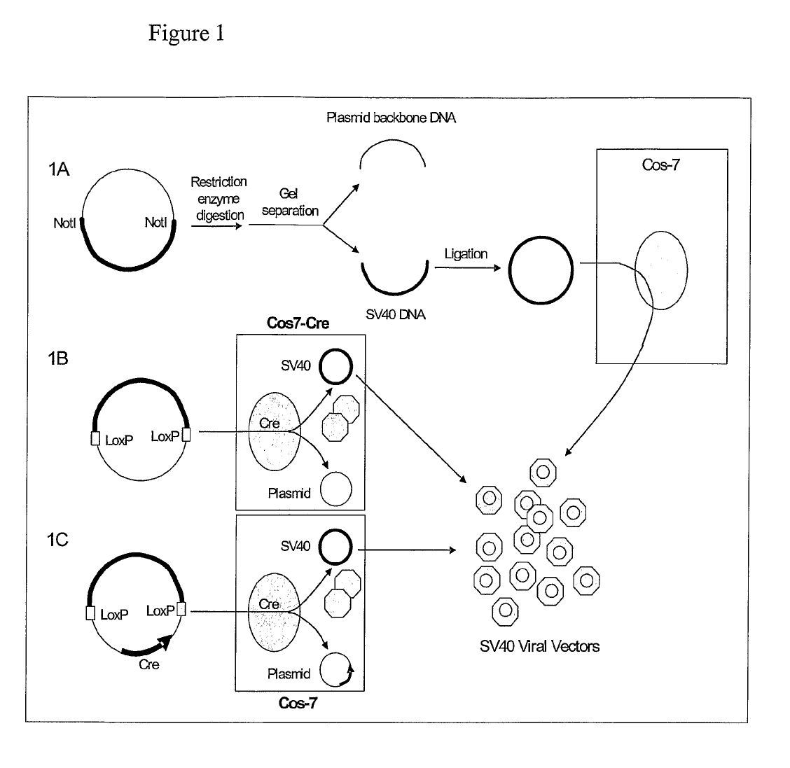

Optionally, the nucleic acid construct is circular in structure. In other embodiments, the nucleic acid construct is linear in structure but capable of allowing recombinase mediated circularization between the target sequences. Of course the construct may also comprise additional nucleic acid sequences (such as the “plasmid” sequences in FIG. 1) attached to one or both target sequences which flank the viral vector. These additional sequence(s) may be considered “non-viral vector” sequences present in the construct.

The construct also optionally further comprises a sequence capable of expressing a recombinase activity, such as cre activity. In some embodiments, the viral vector contains the sequence capable of expressing the recombinase activity. In other embodiments, the sequence is contained within a non-viral (vector) sequence attached to the target sequence.

In additional embodiments, the construct may comprise a cis-element from a viral genome. Non-limiting examples include a viral origin of replication and/or a packaging, or encapsidation, signal. These constructs may be advantageously packaged into a virion or viral particle in the practice of the invention.

In further embodiments, the construct may comprise a heterologous (non-viral) sequence or a heterologous sequence from a different viral genome. The sequence may be capable of expression regulated by sequences in the viral vector portion of the construct. Alternatively, the construct may comprise a heterologous sequence produced or designed based upon a naturally occurring sequence. Non-limiting examples include ribozymes, antisense sequences, shRNAs, miRNAs, and cDNAs. For expression of the sequence, an endogenous promoter of the viral vector may be used. Alternatively, a heterologous promoter may be introduced into the viral vector and used to regulate or direct expression of the heterologous sequence.

In another aspect, a cell comprising a disclosed construct is provided. In some embodiments, the cell is used as a host cell to replicate the construct as a whole. In other embodiments, the cell is a processing cell capable of allowing the construct to be acted on by recombinase activity to form a dsDNA viral vector comprising a target sequence. Generally, the recombinase activity cleaves each target sequence at the same site followed by ligation (or rejoining) of the two ends to result in a single copy of the target sequence. In alternative embodiments, the cell is a packaging cell that allows the construct to be converted to a dsDNA viral vector comprising a loxP sequence which is then packaged into a virion or viral particle. In some embodiments, the packaging cell may express recombinase activity and one or more gene products which facilitate packaging of a dsDNA viral vector comprising a target sequence into a virion or viral particle. As a non-limiting example, the cell may be capable of expressing one or more SV40 encoded gene products, optionally constitutively and so in the absence of a construct of the invention.

In a further aspect, a pair of first and second nucleic acid constructs is provided. The first construct comprises a dsDNA viral vector sequence flanked by target sequences, such as any such constructs described herein. The second construct comprises a sequence capable of expressing a recombinase activity. The two constructs may be used together in a cell comprising both. The cell may be to allow the processing of the first construct to a dsDNA viral vector comprising a single copy of the target sequence. Alternatively, the cell may be to process the first construct and then package the resultant dsDNA viral vector comprising a target sequence into a virion or viral particle as described herein. Of course each of the first and second constructs may comprise additional sequences.

In some embodiments, the second construct is episomal in a cell of the invention. A non-limiting example is where the second construct is present on a plasmid vector. In other embodiments, the second construct is integrated into a cell's genome. Cells containing such a second construct may be considered a host cell. In some embodiments, the cell is capable of expressing one or more SV40 encoded gene products in the absence of the construct or via the second construct.

In an additional aspect, a method of producing a dsDNA viral vector is disclosed. The method may comprise contacting a construct, comprising a dsDNA viral vector sequence flanked by target sequences, with a cognate recombinase activity to excise and circularize the dsDNA viral vector. This method may be performed in vitro or in a cell. Where the method is conducted intracellularly, the recombinase activity may be encoded, and expressed, by a second nucleic acid construct of the invention in the cell. In some embodiments, the cell is also capable of packaging the dsDNA viral vector into virion particles.

The packaged vector is a dsDNA viral vector comprising a single target sequence as described herein. This vector is produced from a dsDNA viral vector sequence flanked by two target sequences (one on either end of the vector sequence) as disclosed herein. The vector may thus contain any sequence that is present in the parental dsDNA viral vector sequence and as flanked by two target sequences. Non-limiting examples include a heterologous sequence present in the parental dsDNA viral vector.

Yet another aspect of the invention is a virion particle comprising a dsDNA viral vector comprising a single target sequence as disclosed herein. The particle may be produced by any method as described herein. In some embodiments, the particle is pseudotyped relative to the viral vector. In other embodiments, the particle is packaged with the same components as normally found with the viral genome from which the viral vector is derived.

The virion particle may be used to deliver the enclosed viral vector to a cell. In some embodiments, this may be by a method comprising contacting the particle with the cell to be infected thereby. In other embodiments, the cell is a target cell which is subject to infection by the particle through the proteinaceous, or non-genetic, surface components of the particle. While the viral vector may be delivered as “naked DNA” to a cell, the use of a particle of the invention advantageously takes advantage of the infectivity provided by the proteinaceous, or non-genetic, surface components of the particle.

Where the viral vector comprises a non-viral or heterologous sequence, the delivery method may be used to introduce that sequence into a cell. This method may comprise contacting a cell with a vector containing a heterologous sequence as described herein.

### EXAMPLES

The conventional means of SV40 vector production is illustrated in FIG. 1A. The SV40 cloning vector that contains a foreign nucleic acid has to be digested with a restriction enzyme to split the vector DNA into two parts: the SV40 DNA and plasmid DNA backbone. The two DNAs are then resolved by agarose gel electrophoresis and the SV40 DNA is purified from the gel. The purified linearized SV40 DNA is re-ligated and the resulting circular DNA is transfected into Cos-7 cells that express T-antigen to complement replication of the SV40 vector.

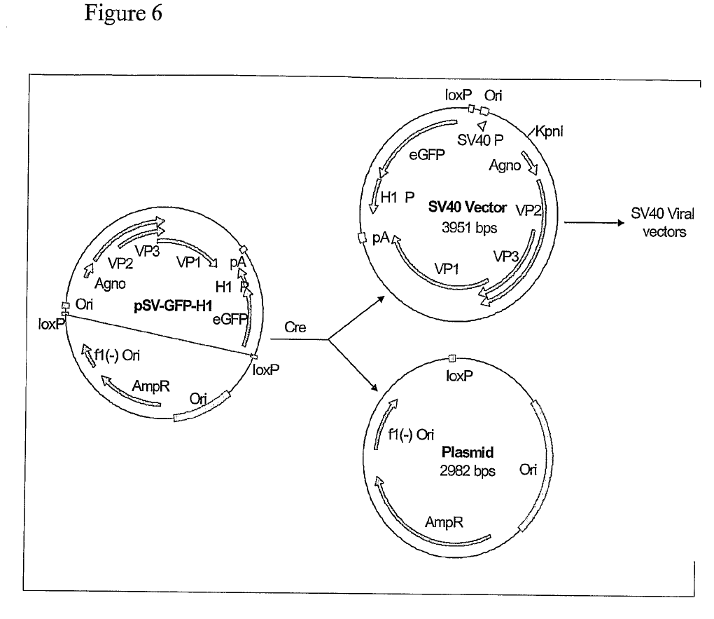

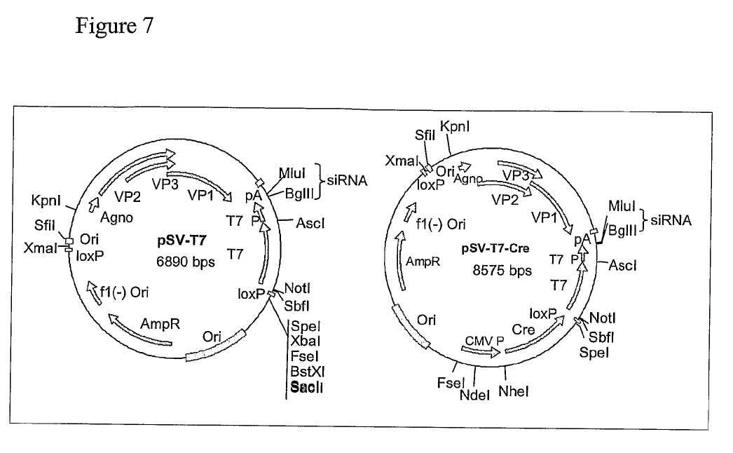

The disclosed invention eliminates the tedious steps of restriction enzyme digestion, agarose gel separation, DNA purification, and religation. Instead, the disclosed invention uses the Cre-Lox recombination system to separate the plasmid DNA backbone from the SV40 vector DNA and to re-circularize both DNAs as shown in FIG. 1B and FIG. 6. Because the SV40 vector DNA contains the SV40 encapsidation signal and the plasmid backbone DNA does not have the encapsidation signal, only the SV40 vector DNA will be packaged into SV40 virion particles. Therefore the disclosed invention streamlines the vector production process as shown in FIG. 1B.

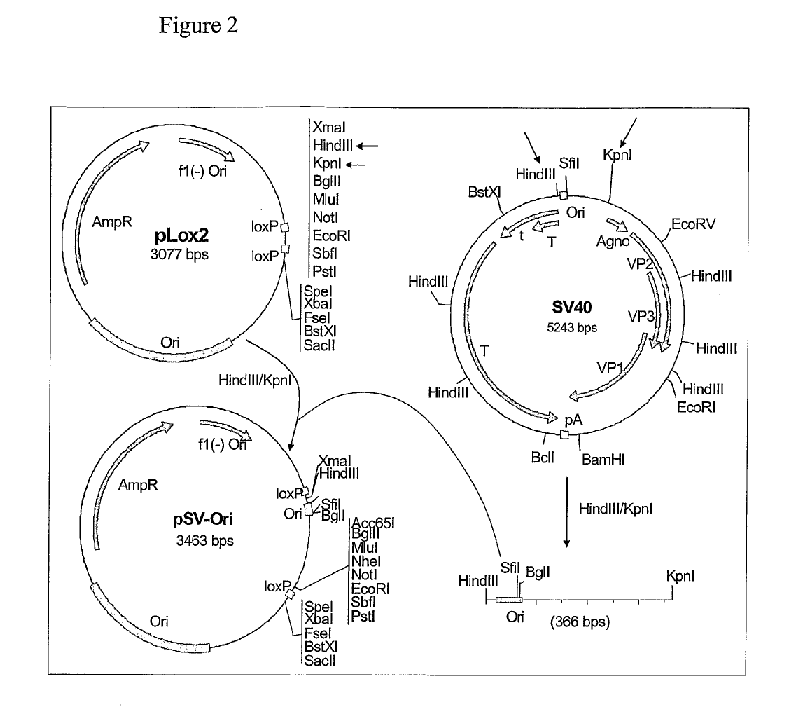

In a first embodiment, referred to as the basic SV40 vector-pSV, the entire replication origin (Ori), capsid genes (VP 1˜3), and the polyadenylation signal (pA) is cloned into a plasmid (pLox2-MCS) that contains two Lox P sites. There are two cloning steps to construct pSV. First (see FIG. 2), the SV40 genome (strain 776) is digested with KpnI and HindIII. The resulting 366 bp fragment that encompasses the SV40 replication origin, the early and late promoters, and the leader sequence of the late genes is isolated and cloned into the HindIII/KpnI sites of pLox2-MCS. The resulting construct is pSV-Ori (FIG. 2).

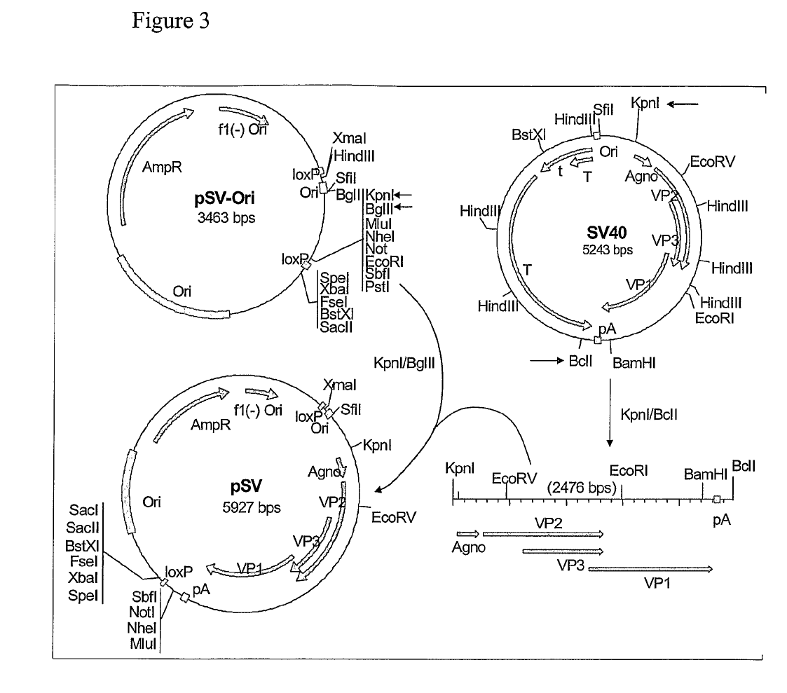

Next (see FIG. 3), the SV40 genome is digested with KpnI and BclI. The resulting 2476 bp fragment that encompasses the late genes (agno, VP1, VP2, and VP3) and the polyadenylation signal is cloned into the corresponding sites of pSV-Ori. The resulting construct is the basic SV40 vector (pSV, FIG. 3) that allows future cloning of foreign nucleic acids into the multiple cloning sites (MCS) that contains MluI, NheI, NotI, and SbfI sites. Viral vectors can be produced simply by transfecting Cos7-Cre complement cell line with pSV-based vector DNA as shown in FIG. 1B.

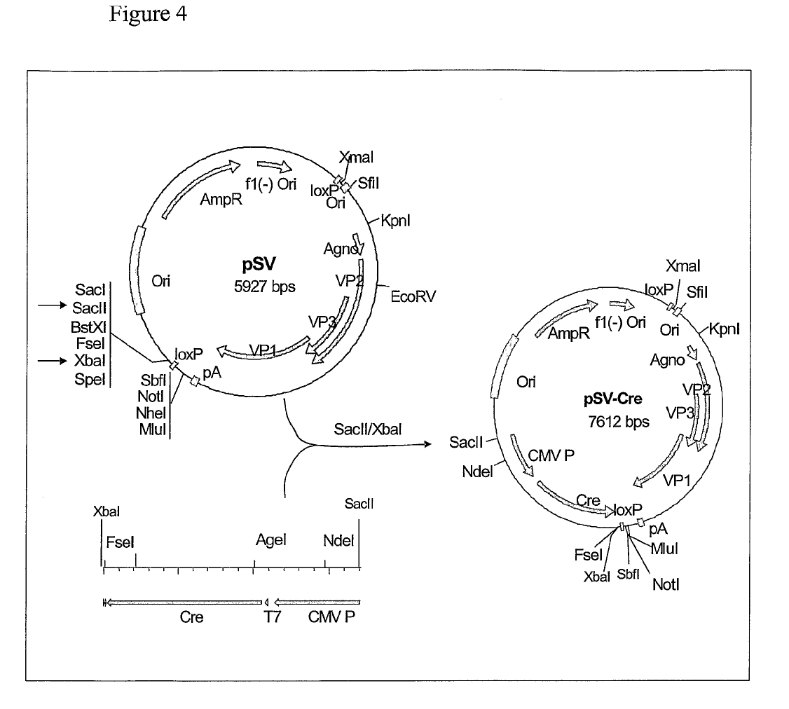

Another embodiment of the invention is SV40 vector pSV-Cre (FIG. 4). This vector construct is derived from the parental basic SV40 vector pSV by adding a Cre gene that is expressed from a eukaryotic promoter. The promoter can be any suitable eukaryotic promoter, including cytomegalovirus (CMV) immediate-early promoter and elongation factor 1-α (EF-1α) promoter as non-limiting examples. Viral vectors can be produced simply by transfecting Cos-7 cells with pSV-Cre-based vector DNA as shown in FIG. 1C.

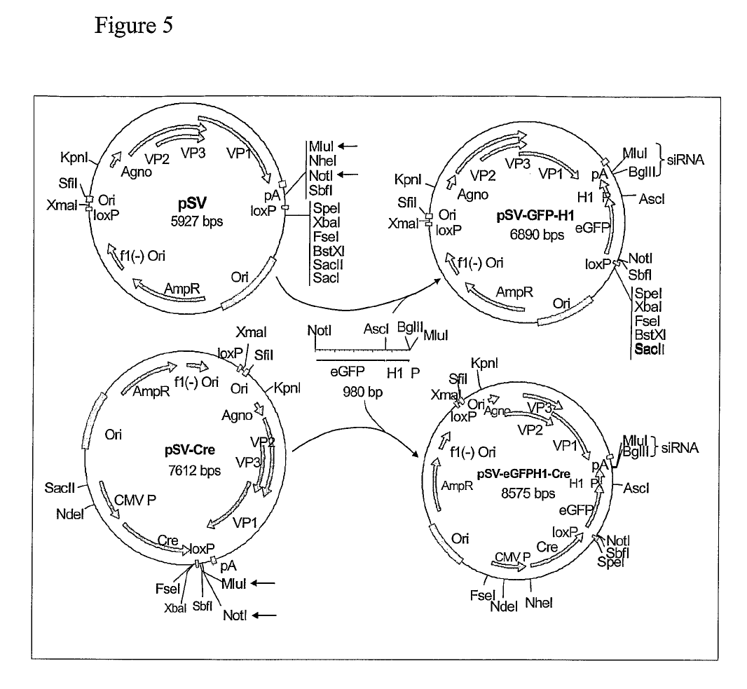

Any cDNA, such as GFP, or DNA fragment encoding micro RNA (miRNA) or antisense DNA can be cloned into cloning sites between the polyadenylation signal and the immediate downstream Lox P site of either pSV or pSV-Cre in an orientation opposite to the SV40 late genes (VP 1˜3) (FIG. 5). After homologous recombination mediated by Cre the vector plasmid is split into two circular DNAs (FIG. 6). This results in positioning the cDNA or the miRNA DNA or the antisense DNA immediately downstream of the SV40 early promoter in the circular SV40 vector DNA that contains the SV40 replication origin and encapsidation signal and the late genes. Therefore the expression of the introduced cDNA or miRNA DNA or antisense DNA is driven from the SV40 early promoter (FIG. 6).

T-antigen expressed in Cos-7 cells can activate the SV40 replication origin and replicate the SV40 vector genomes. Large amount of capsid proteins (VP1, VP2, and VP3) are expressed from the SV40 late promoter during replication of the SV40 vector genome and package the circular SV40 vector genomes into virion particles. Another circular DNA after the Cre-mediated homologous recombination is derived from the plasmid backbone. Since there is no SV40 encapsidation signal, the circular plasmid backbone is not packaged into virion particles.

Another embodiment of the invention is an SV40 vector for expression of short hairpin RNA (shRNA) or ribozyme. Any Pol III promoter (such as H1, U6, and tRNA promoter, EBER I, and EBER II and any regulatable Pol III promoter) as a non-limiting example can be cloned into the multiple cloning sites located in between the polyadenylation signal and the immediate downstream Lox P site of either pSV or pSV-Cre. A reporter or marker gene, such as eGFP can be inserted under the control of the Pol II promoter for future vector titering purposes (FIG. 5, 6). Oligonucleotides that encode shRNA or a ribozyme can be cloned into the cloning sites downstream of the Pol III promoter. Viral vectors are produced following transfection of Cos-7 or Cos7-Cre cells and can be titered by the number of GFP-positive cells after transduction.

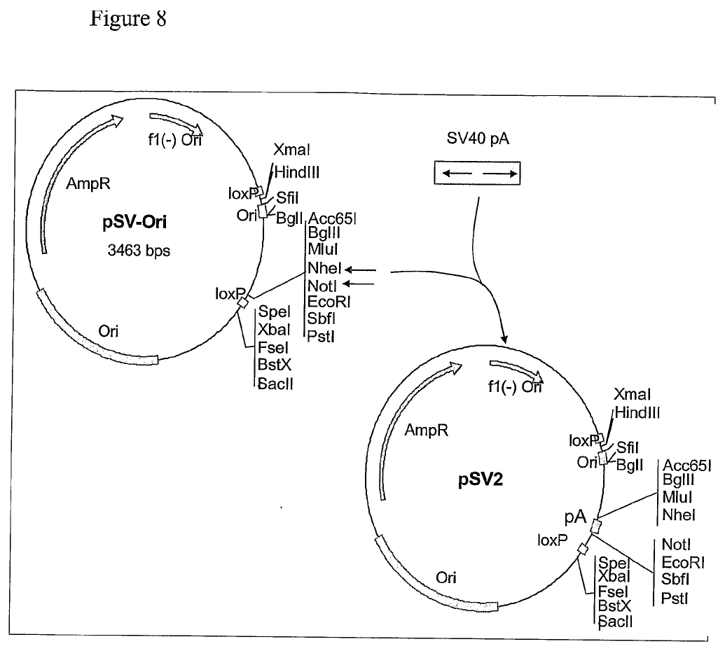

Another embodiment of the invention is to use T7 polymerase and T7 promoter for expression of shRNA, miRNA, ribozyme, and cDNA via an SV40 vector. A DNA fragment that encodes T7 polymerase followed by a T7 promoter is cloned into the multiple cloning sites located in between the polyadenylation signal and the immediate downstream Lox P site of either pSV or pSV-Cre (FIG. 8). Oligonucleotides that encode the “pay-loads” can be cloned into the cloning sites downstream of the T7 promoter. After Cre-mediated homologous recombination, T7 polymerase is expressed under the control of the SV40 early promoter as described above. T7 polymerase binds to T7 promoter and strongly activates the promoter. This results in strong expression of the pay-loads.

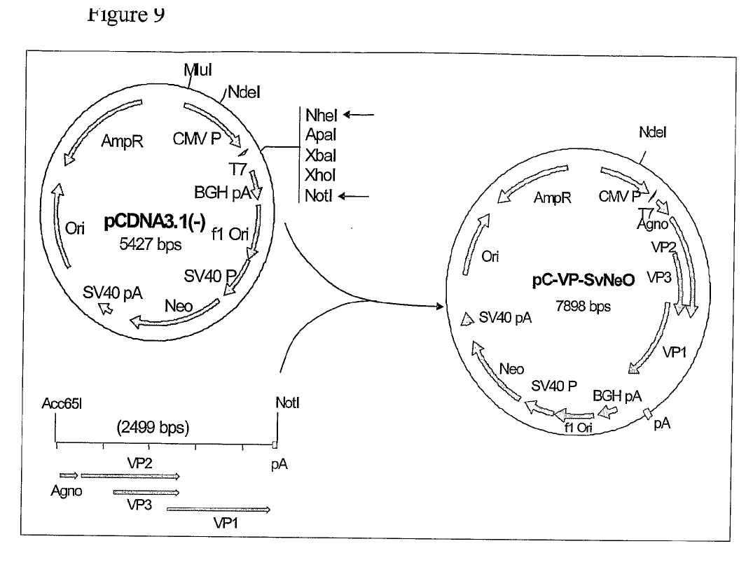

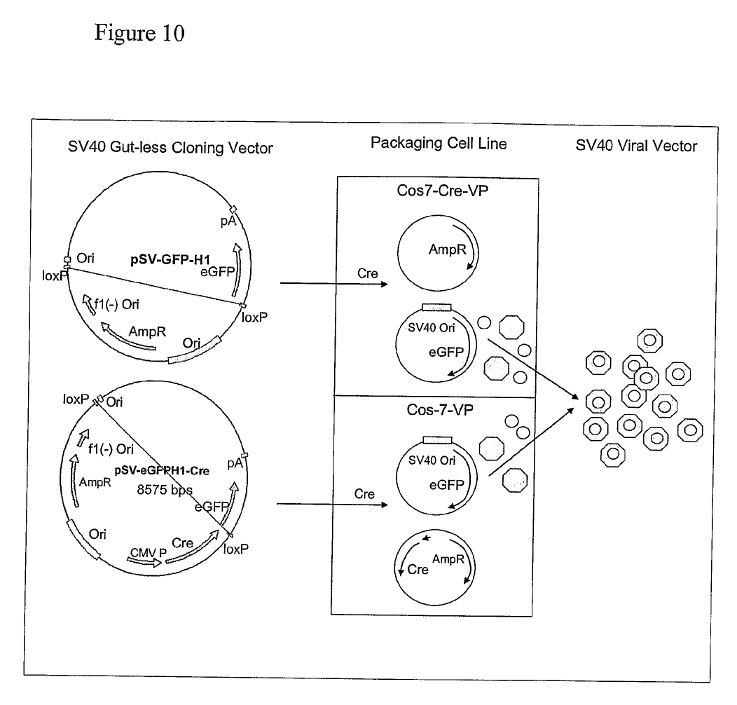

Another embodiment of the invention is a “gut-less” SV40 vector as a representative example of a minimal vector with only the necessary cis components from a viral genome. A DNA fragment that encompasses the SV40 early and late polyadenylation signal is cloned into NheI site of pSV-Ori (FIG. 2). The resulting pSV2 (FIG. 9) is a gut-less SV40 cloning vector. The gut-less vector contains only the cis-elements for SV40 DNA replication and encapsidation and allows insertion of foreign DNA up to 5 kb in size. Because there is no SV40 viral gene in the gut-less SV40 cloning vector a complement cell line Cos7-Cre-VP is constructed to produce the SV40 gut-less viral vector. A plasmid pC-VP-SvNeo is made by cloning a DNA fragment that encompasses SV40 agno, VP1, VP2, and VP3 genes downstream the CMV promoter of pCDNA3.1 (−) (FIG. 10). Cos-7 and Cos7-Cre cells are transfected with pC-VP-SvNeo and selected by G418. The resulting G418-resistant cell clones, Cos7-VP and Cos7-Cre-VP are analyzed by Western blot analysis using anti-VP antibody. The highest VP-expression clone is selected as the SV40 gut-less vector packaging cell line.

Another embodiment of the invention is the application of the SV40 vectors described herein. These vectors can be used to deliver any nucleic acid sequence, including cDNA, antisense, ribozyme, and small interfering RNA (siRNA) as non-limiting examples, into cells for the purposes of drug screening and gene therapy.

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

1. A nucleic acid construct comprising a dsDNA viral vector sequence flanked by target sequences of an enzyme-mediated sequence-specific homologous recombination system.

2. The construct of claim 1 wherein the target sequences are selected from loxP sequences, sequences recombined by the FLP recombinase, and sequences recombined by the XerD recombinase.

3. The construct of claim 1 or 2 further comprising a sequence capable of expressing a cre recombinase activity.

4. The construct of claim 1 or 2 or 3 wherein said vector sequence comprises a viral origin of replication and a packaging or encapsidation signal.

5. The construct of claim 1 or 2 or 3 or 4 wherein said dsDNA viral vector is an SV40 viral vector or a vector derived from genus polyomavirus.

6. The construct of claim 1 or 2 or 3 or 4 or 5 wherein said dsDNA viral vector is capable of expressing a heterologous (non-viral) sequence.

7. A cell comprising the construct of claim 1 or 2 or 3 or 4 or 5 or 6.

8. A pair of first and second nucleic acid constructs, said first construct comprising a dsDNA viral vector sequence flanked by loxP sequences and said second construct comprising a sequence capable of expressing a cre recombinase activity.

9. A cell comprising the constructs of claim 8.

10. The cell of claim 9 wherein said second construct is episomal or is integrated into the host cell genome.

11. The cell of claim 7 or 9 or 10 wherein said dsDNA viral vector is an SV40 vector.

12. The cell of claim 7 or 9 or 10 or 11 wherein said dsDNA viral vector is capable of expressing a heterologous (non-viral) sequence.

13. The cell of claim 7 or 9 or 10 or 11 or 12 wherein said cell is capable of expressing one or more SV40 encoded gene products in the absence of said constructs.

14. A method of producing a dsDNA viral vector, said method comprising contacting a construct of claim 1 with a CRE recombinase activity to excise and circularize said dsDNA viral vector.

15. The method of claim 14 wherein said contacting occurs intracellularly.

16. The method of claim 14 or 15 wherein said CRE recombinase activity is encoded by a second nucleic acid construct.

17. The method of claim 15 wherein said cell is capable of packaging said dsDNA viral vector into virion particles.

18. The method of claim 14 or 15 or 16 or 17 wherein said dsDNA viral vector is an SV40 viral vector.

19. A dsDNA viral vector comprising one copy of a target sequence of an enzyme-mediated sequence-specific homologous recombination system.

20. The vector of claim 19 wherein said sequence is loxP, or that for the FLP recombinase, or that for the XcrD recombinase.

21. The vector of claim 19 or 20 wherein said vector is SV40 based.

22. The vector or claim 19 or 20 or 21 further comprising a non-viral (heterologous) sequence.

23. A virion particle comprising the vector of claim 19 or 20 or 21 or 22.

24. A method of delivering a non-viral or heterologous sequence to a cell, said method comprising contacting said cell with a vector of claim 22 or a particle of claim 24.
